(19) United States
(12) Patent Application Publication
(10) Pub. No.: US 2009/0254090 A1
(43) Pub. Date: Oct. 8, 2009

(54) COMPRESSION STAPLE

(75) Inventor: Emmanuel Lizee, Saint Ismier (FR)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: TORNIER SAS, Saint Ismier (FR)

(21) Appl. No.: 12/417,492

(22) Filed: Apr. 2, 2009

(30) Foreign Application Priority Data
Apr. 2, 2008 (FR) 08 52184

(51) Int. Cl. A61B 17/064

(52) U.S. Cl. 606/75

(57) ABSTRACT

A compression staple according to an embodiment of the present invention includes two arms connected by a transverse bridge. Each arm includes an anchor portion and a manipulation portion arranged so as to extend from one another and connected by a separation mechanism or breakaway feature. The anchor portion is used to anchor the arm in a bone while the manipulation portion is used to guide the arm in displacement relative to the bridge.

### CROSS-REFERENCE TO RELATED APPLICATION

This application claims foreign priority to French DEMANDE DE BREVET D'INVENTION number 08 52184, filed with the Institut National de la Propriété Industrielle on Apr. 2, 2008, and entitled, “AGRAFE DE COMPRESSION,” which is incorporated herein by reference in its entirety for all purposes.

### TECHNICAL FIELD

Embodiments of the present invention relate generally to the spanning of bone elements, and more particularly to compression staples.

### BACKGROUND

A compression staple is used to hold two parts of a bone in place relative to one another so that they can be joined together. A compression staple typically includes two arms connected by a transverse bridge, the arms being inserted into the bone on either side of a fracture or osteotomy of a bone to be repaired. A staple of this type further comprises means for moving the two arms towards one another, which in turn makes it possible to press the two bone parts against one another with a specific amount of pressure.

In particular, EP-A-0 955 011 illustrates a compression staple in which the arms are moved towards one another once the staple has been implanted by deforming the transverse bridge of the staple. In this device the bridge of the staple forms an eyelet, the deformation of which causes it to open out and extend perpendicular to the axis of the bridge, which in turn makes it possible to shorten the bridge and move the arms towards one another. However, once deformed, the size of the bridge extending perpendicular to the plane defined by the arms of the staple is substantial. The size of the bridge extending perpendicular to the plane of the arms when the staple is implanted in a bone is further emphasized by the cylindrical shape of the bone and may often cause discomfort beneath the skin and lead to the tissue next to the bone becoming damaged. Furthermore, the bridge is deformed by exerting an axial pressure on the ends of the arms which emerge from the bone matter. For this purpose, these ends of the arms are configured so as to clearly extend above the surface of the bone. Projection of the staple relative to the surface of the bone increases the risk of causing discomfort beneath the skin and damaging tissue.

### SUMMARY

Embodiments of the present invention relate to a compression staple of the type comprising two arms connected by a transverse bridge, characterized in that each arm comprises a manipulation portion and an anchor portion arranged so as to extend from one another and connected by a breakaway feature, the anchor portion being used to anchor the arm in a bone while the manipulation portion is used to guide the arm in displacement relative to the bone.

Embodiments of the present invention include a compression staple which is flat and compact when said staple is implanted in a bone. According to some embodiments of the present invention, the manipulation portion is used to guide the arm in displacement relative to the bridge and parallel to a median plane defined by the longitudinal axis of the arm and the longitudinal axis of the bridge. The bridge may be connected to each arm at an end of the manipulation part adjacent to the anchor part, and the manipulation and anchor parts of each arm are connected by a separation means, or breakaway feature, which permits the manipulation portion to be broken off by exerting a force transverse to a median plane defined by the longitudinal axis of the arm and the longitudinal axis of the bridge. In this way, a force exerted on either the manipulation or anchor portions breaks the manipulation portion away from the anchor portion while the other part of the arm remains still.

The separation means of each arm includes a separable bridging connection, or breakaway feature, between the manipulation and anchor parts, the breakaway feature having a thickness in a direction transverse to the median plane which is smaller than the thickness of the manipulation and/or anchor parts in the same direction, according to embodiments of the present invention. In some cases, the separation means or breakaway feature includes at least one notch in the arm of the staple. In other cases, the separation means define a cutting face between the manipulation and anchor elements of the arm of the staple. The cutting face of each arm may be arranged so the anchor part of the arm is inclined towards the anchor part of the other arm at the same height as at least a part of an edge of the bridge on the side of the manipulation part of each arm. According to some embodiments, the end of the anchor part of each arm adjacent to the manipulation part of the arm has a cross-section, perpendicular to the longitudinal axis of the arm, which is greater than the cross-section of the rest of the anchor part perpendicular to the longitudinal axis of the arm.

According to some embodiments of the present invention, before the staple is implanted in a bone, the bridge is curved outwards on the side of the anchor parts of the two arms. In some cases, the staple may be formed of a metal plate cut substantially into an H shape, each arm of the H being able to pivot relative to the transverse bridge of the H and parallel to the median plane of the plate. According to some embodiments of the present invention, the second part of each arm includes an aperture for receiving an element for actuating displacement of the arm relative to the bridge and parallel to a median plane defined by the longitudinal axis of the arm and the longitudinal axis of the bridge. In some cases, the anchor part of each arm includes a means for holding the anchor part in place when anchored in a bone.

A method for fitting and placing a compression staple as described above so as to join together two bone parts includes drilling a hole in each of two bone parts, inserting the anchor portion of one arm of the staple into the hole in one of the bone parts, inserting the anchor portion of the other arm of the staple into the hole in the other bone part until the edge of the bridge that faces the anchor portions contacts the bone surface, inclining the anchor portions of the two staple arms toward one another by exerting a diverging force on the manipulation portion of at least one of the arms relative to the manipulation portion of the other arm, and separating the manipulation portion of each arm of the staple by breaking the manipulation portion off at the breakaway feature or separation means. The holes may be drilled in the two bone parts abutting one another with a distance between the holes which is greater than or equal to the center-to-center distance between the anchor parts of the staple arms when the anchor portions are parallel. In some cases, before the anchor parts of the arms of the staple are inserted into the holes in the bone parts, the anchor parts of the two arms are diverted away from one another by exerting on the manipulation part of at least one of the arms a converging force towards the manipulation part of the other arm.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a perspective view of a compression staple, according to embodiments of the present invention;

FIG. 2 is front view of the compression staple of FIG. 1, according to embodiments of the present invention;

FIG. 3 is an enlarged detail view of the breakaway feature of the compression staple of FIGS. 1 and 2, taken along arrow III in FIG. 1, according to embodiments of the present invention;

FIG. 4 is a front view of the compression staple of FIGS. 1 to 3 during a first step of implanting the staple in a bone, according to embodiments of the present invention;

FIG. 5 is a front view of the compression staple of FIGS. 1 to 4 during a second step of implanting the staple in a bone, according to embodiments of the present invention; and

FIG. 6 is a front view of the compression staple of FIGS. 1 to 5, in which the staple is implanted in a bone, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

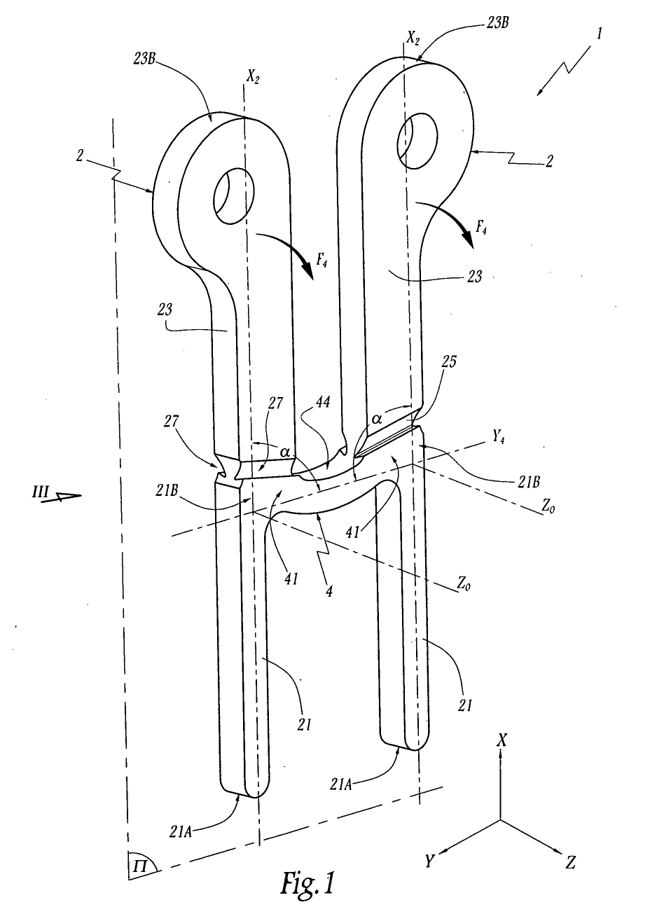

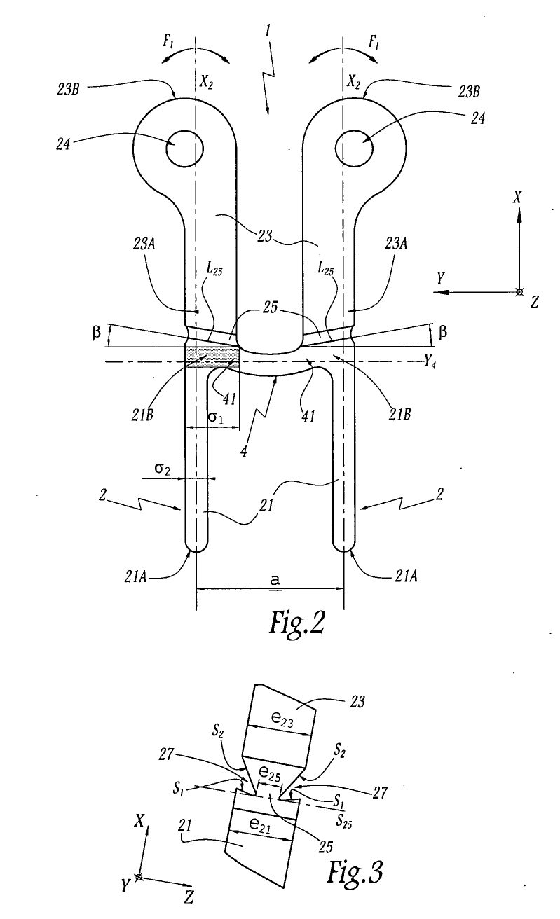

The compression staple 1 shown in FIG. 1 comprises two elongate arms 2 and a transverse bridge 4 connecting the two arms. The staple 1 may be formed by a stainless steel plate cut basically into an H shape so as to define the two arms 2 and the bridge 4 connected to a central part of each arm 2. In a variation, the staple 1 may be made of any other suitable material, for example titanium. The staple 1 joins together two parts of a bone following a fracture or osteotomy. It can be seen that X is a longitudinal direction of the staple 1, Y is a transverse direction of the staple 1, and Z is a depth direction of the staple 1. Before it is implanted in a bone, the staple 1 is in an initial configuration shown in FIGS. 1 and 2, in which the longitudinal axes X₂ of the two arms 2 are substantially parallel to one another and to the direction X and are perpendicular to the longitudinal axis Y₄ of the bridge 4, which in turn is parallel to the direction Y.

Due to the mechanical properties of the material of which the staple 1 is made, each arm 2 of the staple 1 can be displaced by being pivoted on either side relative to the adjacent end 41 of the bridge 4 from its perpendicular position relative to the bridge 4 shown in FIGS. 1 and 2, as shown by the double-headed arrows F₁ in FIG. 2, while remaining within a median plane π of the staple 1 defined by the longitudinal axis X₂ of each arm 2 and the longitudinal axis Y₄ of the bridge 4. Each arm 2 can thus be pivoted relative to the bridge 4 about an axis Z₀ perpendicular to the median plane π, while remaining in said median plane π, between a position in which the angle α delimited between the longitudinal axis Y₄ of the bridge 4 and the longitudinal axis X₂ of the arm 2 is approximately 80° and a position in which the angle α is approximately 110°.

Each arm 2 of the staple 1 includes two elongate parts arranged so as to extend from one another: a part 21 to be anchored in a bone and a guiding part 23 to be actuated so as to pivot the arm 2 relative to the bridge 4 and parallel to the median plane π. Part 21 may be referred to as an anchor portion 21 of the arm 2, and part 23 may be referred to as a manipulation portion 23 of the arm 2. According to embodiments of the present invention, the two parts extending from one another may be arranged so as to be aligned or at an angle from one another. The parts 21 and 23 are connected by a separable bridging connection or breakaway feature 25, which connects an end 21 B of the part 21 to an end 23A of the part 23. The thickness e₂₅ of the separable bridging connection 25 in the depth direction Z of the staple 1 is smaller than the thickness e₂₁ of the part 21 and the thickness e₂₃ of the part 23 in the direction Z. In particular, in this embodiment the thicknesses e₂₁ and e₂₃ of the parts 21 and 23 are the same, whereas the thickness e₂₅ of the separable bridging connection 25 is less than half the thickness e₂₁ or e₂₃ of the parts 21 and 23. The bridging connection 25 may thus be broken off by a force F₄ exerted in the depth direction Z of the staple 1; in other words, the part 23 may be separated from the part 21 at the breakaway feature 25 by exerting a force F₄ transverse to the median plane π and exerted in the vicinity of the free end of one of the parts 21 or 23 of the arm while the other part remains still. As shown in FIG. 1, the force F₄ is exerted in the vicinity of the free end 23B of each part 23, whereas each of the parts 21 remains still.

Each of the two ends 41 of the transverse bridge 4 of the staple 1 is connected to the end 21B of the part 21 of a arm 2 in such a way that each end 41 of the bridge 4 is adjacent to the separable bridging connection 25 of an arm 2. As can be seen in FIGS. 1 and 2, before the staple 1 is implanted in a bone, the bridge 4 of the staple 1 is curved outwardly toward the parts 21 of the arms 2 to be anchored in a bone. In other words, an edge 44 of the bridge 4 is concave directed towards the parts 23 and is convex towards the parts 21.

According to some embodiments of the present invention, the breakaway feature 25 of each arm 2 is delimited by two point-shaped notches 27 which are symmetrical to one another about the median plane π of the staple and grooved in the depth direction Z of the arm 2. As shown in FIG. 3, each notch 27 defines two inner faces S₁ and S₂ of the arm 2 which are adjacent to the part 21 and the part 23 of the arm 2 respectively. The two faces S₂ of an arm 2 are inclined towards the part 21 of the arm at an angle of approximately 45° relative to the longitudinal axis X₂ of the arm, whereas the two faces S₁ of an arm 2 lie substantially within the same plane which, in the initial configuration of the staple 1, is parallel to the direction Z of the staple 1 and is inclined at an angle β of approximately 15° relative to the direction Y of the staple. The cutting face S₂₅ of the separable bridging connection 25, which corresponds to the smallest cross-sectional depth e₂₅ of the bridging connection 25, also lies in a plane parallel to the direction Z and is inclined at an angle β of approximately 15° relative to the direction Y when the staple 1 is in its initial configuration.

The trace line L₂₅ of the cutting face S₂₅ of each arm 2 can be seen in FIG. 2. When the staple 1 is in the initial configuration, each trace line L₂₅ is inclined at an angle β relative to the direction Y of the staple in such a way that the trace lines L₂₅ of the cutting faces S₂₅ of the two arms 2 are inclined towards the bridge 4 and form a continuation of the edge 44 of the bridge 4 on the side of the parts 23 of the arms 2. A configuration of this type of the cutting faces S₂₅ in the initial configuration of the staple 1 ensures that, when the two arms 2 are inclined relative to their starting position so as to move the parts 21 towards one another, the cutting face S₂₅ of each arm 2 lies in a plane substantially parallel to the directions Y and Z of the staple, this plane being arranged at the same height as the ends of the edge 44 of the bridge 4, as shown in FIG. 5.

As shown in FIG. 2, the end 21B of the part 21 of each arm 2 has a cross-section σ₁, taken perpendicular to the longitudinal axis X₂ of the arm, which is greater than the cross-section σ₂, taken perpendicular to the axis X₂, of the rest of the part 21. The part 21B, which connects the bridge 4 to the part 21 of each arm 2 and is subjected to significant stresses when the staple is implanted, is thus reinforced and able to withstand local strains caused by the pivoted displacement of the arms 2 relative to the bridge 4. According to some embodiments of the present invention, the cross-section of the part 23 of each arm 2 is selected to be equal to the cross-section σ₁ of the end 21B such that each arm 2 cannot be deformed at the connection between the parts 21 and 23 when the arm 2 is pivoted by applying a force F₂, F₃ to the free end 23B of the part 23. In order to apply a force F₂, F₃ of this type for pivoting arm 2, the part 23 of each arm 2 is provided with, in the vicinity of its free end 23B, an aperture 24 able to receive an element for pivoting the arm.

A method for fitting the compression staple 1 to join together two bone parts 9 of a bone 10 which has been fractured or has undergone osteotomy, for example a phalange or metatarsal, includes one or more of the following steps, according to embodiments of the present invention.

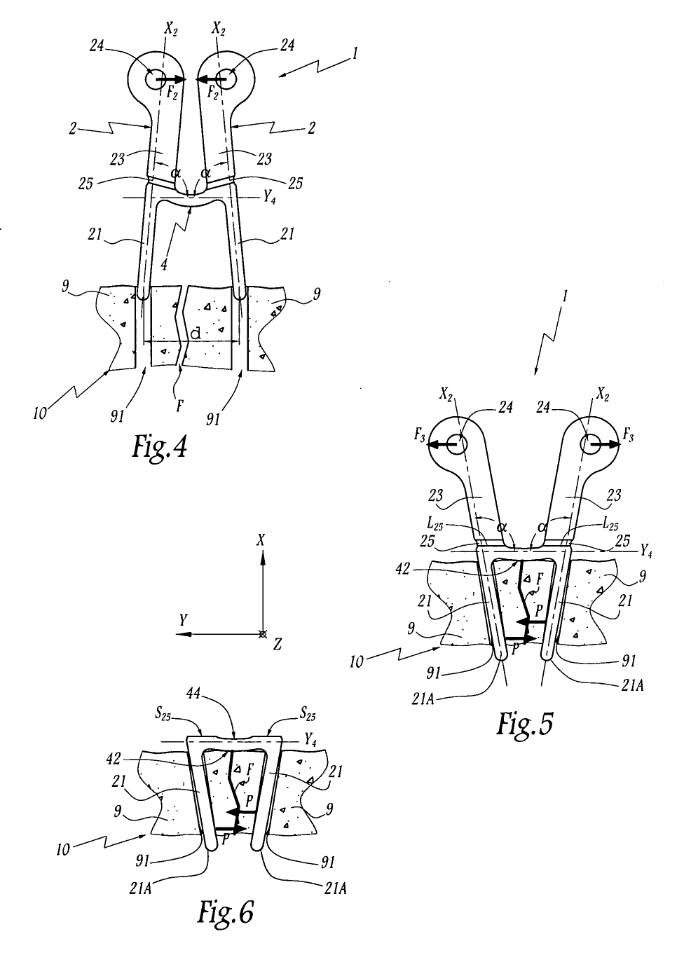

A hole 91 is first drilled in each of the two bone parts 9 on either side of the site F of the fracture or osteotomy of the bone 10. When the bone parts 9 abut one another with no compression, as shown schematically in FIG. 4, the holes 91 are advantageously spaced from one another at a distance d which is greater than the center-to-center distance a between the parts 21 of the arms 2 in the initial configuration of the staple 1, in which the arms 2 are parallel.

The parts 21 of the arms 2 of the staple are then inserted into the holes 91. For this purpose, each of the arms 2 is pivoted relative to the bridge 4 and parallel to the median plane π so as to move the parts 21 of the two arms 2 away from one another and away from the bridge 4 by applying a converging force F₂ on the free ends 23B of the two arms 2 at the apertures 24. The parts 21 are thus arranged in a diverging configuration in which the free ends 21A are spaced apart by the distance d. The parts 21 can then be inserted into the holes 91 in the bone parts 9.

By moving the parts 21 away from one another, the parts 21 can be inserted into the holes 91 in the bone parts 9. However, this diverging movement may be stopped as soon as the parts 21 penetrate the holes 91. According to a variation (not shown), the holes 91 may be drilled so as to be spaced apart, when the bone parts 9 abut one another, at a distance d which is equal to the center-to-center distance a between the parts 21 of the arms 2 when the staple 1 is in the initial configuration (e.g. when the arms 2 are substantially parallel). In this way, forming the holes 91 at a particular distance prevents or minimizes the need to move the parts 21 away from one another before they are inserted into the holes 91.

When the parts 21 are inserted into the holes 91, the staple 1 gradually returns, for example under the action of a tool which is used in the apertures 24, to its initial configuration, for example a configuration in which the arms 2 are parallel. This return to the initial configuration of the staple 1 is triggered when the parts 21 are sunk into the holes 91. The parts 21 are lowered until an edge 42 of the bridge 4 on the side of the parts 21 contacts the surface of the bone 10. As the parts 21 are inserted into the holes 91, the bone parts 9 are pushed more firmly against one another as the arms 2 return toward a configuration in which they are parallel.

Once the parts 21 have been sufficiently lowered into the holes 91 for the edge 42 of the bridge 4 to contact the surface of the bone 10, the arms 2 are again pivoted relative to the bridge 4 and parallel to the median plane π in such a way that the parts 21 are inclined towards one another and away from the bridge 4. For this purpose, a diverging force F₃ is applied to the free ends 23B of the parts 23 at the apertures 24. As the free ends 21A of the parts 21 are moved towards one another, a force P reconciling the bone parts 9 is produced and this makes it possible to urge the faces of the two bone parts against one another at the site F with a specific and desired level of pressure. The parts 21 of the arms 2 remain inclined towards one another despite the reaction force exerted by the wall of each hole 91 on the corresponding part 21, according to embodiments of the present invention.

The part 23 of each arm 2 is then separated from the adjacent part 21 by exerting a pushing and/or pulling force transverse to the median plane π of the staple 1 on the free end 23B of the part 23, as shown by the arrow F₄ in FIG. 1. The pulling and/or pushing force F₄ is exerted on the part 23 of the arm 2 until the part 23 breaks off from the part 21 at the breakaway feature 25 of the arm 2. Once the part 23 of each arm 2 has been snapped off the corresponding part 21, the staple 1 is in the implanted configuration shown in FIG. 6, in which each cutting face S₂₅ is substantially parallel and planar with the plane of the edge 44 of the bridge 4 which was deformed when the compression force was applied to the bone parts 9.

As can be seen in FIG. 6, when the staple 1 is implanted in the bone 10, for example when the parts 23 have been separated from the parts 21 of the arms, the staple 1 is arranged flat and compact against the surface of the bone. According to some embodiments of the present invention, after the staple 1 is implanted in the bone 10, the staple 1 is substantially flush with the surface of the bone. According to some embodiments of the present invention, the surface of the staple 1 remote from the bone 10 and formed by the edge 44 of the bridge 4 and the cutting faces S₂₅ of the parts 21 include no projections or protrusions. In particular, the features of the separable bridging connections 25 of the arms 2, in particular with regard to inclination, are adapted such that no projections from the upper edge 44 of the bridge 4 remain once the parts 23 have been snapped off and the parts 21 have been moved towards one another. The provision of separable parts 23, which guide the arms 2 in displacement relative to the bridge 4, means that the bridge 4 itself does not have to be used to exert a reconciliation force on the arms. Consequently, the size of the bridge 4 at the surface of the bone may be minimal, which reduces the risks of discomfort beneath the skin and deterioration of the tissue in the vicinity of the staple.

Furthermore, as can be seen in the example described above, it is possible to control the compression load applied to the bone parts 9 by applying a suitable diverging force F₃ to the parts 23 of the arms 2. It is also possible to reverse the converging movement of the parts 21 when applying compression to the bone parts 9, in particular if the compression produces an undesired displacement. This controlled deformation of the staple 1 according to embodiments of the present invention differs from the deformation obtained with existing staples made of shape-memory material, in which the compression load applied to the bone parts to be joined together is determined by the features of the shape-memory material and cannot be controlled once the staple is implanted. Furthermore, a compression staple according to embodiments of the present invention made, for example, of stainless steel or titanium is less likely to provoke allergic reactions than a staple made of shape-memory material and does not require specific temperature conditions during storage and use.

In addition, due to the provision of the parts 23 of the arms 2 which guide the arms 2 as they pivot in the median plane π of the staple relative to the bridge 4, compression may be applied to the bone parts when the staple 1 has already been lowered into the bone to the maximum extent, for example when the bridge 4 is in contact with the surface of the bone. Consequently, it is not necessary to further impact the staple once the arms have been brought together, which is advantageous. Lastly, a compression staple according to embodiments of the present invention made, for example, of stainless steel may be easily produced by laser cutting, waterjet cutting, or the like with minimal production costs.

The invention is not limited to the examples described and shown. In particular, a compression staple according to embodiments of the present invention may have a different shape from that shown in the figures. The bridge 4 may, in particular, be rectangular instead of being curved outwardly toward the parts 21 of the arms. The proximal end of the part 21 of each arm 2 to be anchored in a bone may also have a larger cross-section so as to prevent the arm from bending, according to embodiments of the present invention.

Furthermore, the separable means (also referred to as the breakaway features) connecting the manipulation parts and the anchor parts of a compression staple according to embodiments of the present invention may be of any suitable type other than a separable bridging connection. If the separable means are formed by a separable bridging connection, said bridging connection may also have features which are different from those of the bridging connection 25 described above, for example in terms of location or inclination relative to the arms. For example, instead of including a reduced thickness, groove, and/or notch, the breakaway feature may have geometry resembling the surrounding parts of the arm but may include a localized weakness in the material of the arm at the location of the breakaway feature, to facilitate separation of the manipulation portion from the anchor portion.

A compression staple according to embodiments of the present invention may also be made of any suitable material other than stainless steel. In particular, a compression staple having separable parts 23 for guiding the arms when pivoted relative to the bridge may be made of a shape-memory material, according to embodiments of the present invention. The provision of the parts 23 thus compensates for any possible return of the shape-memory material towards a converging configuration of the parts 21 in the case of temperature variations before the staple is implanted, according to embodiments of the present invention.

In addition, it is possible to omit some steps of the methods described above for fitting or placing a staple according to embodiments of the present invention. In particular, a compression staple according to embodiments of the present invention may be implanted in a bone in its initial configuration, with no prior diverging movement of the parts 21 of its arms. The distance d between the holes in the bone parts may then be selected so as to be substantially equal to the center-to-center distance a between the parts 21 in the initial configuration of the staple. Furthermore, depending on the shape of the bone parts to be brought together, it may not be possible to incline the parts 21 of the arms towards one another when applying the compression force to the bone parts. In this case, a diverging force F₃ is nevertheless applied to the parts 23 of the arms so as to incline the parts 21 towards one another.

According to an alternative embodiment of the present invention (not shown), a compression staple may also be provided with means for holding the parts 21 of the staple in place when anchored in the bone parts so as to stop the staple from exiting the bone. For example, the ends of anchor portions 21 may include a hook, a barb, a tine, an arrowhead, a tooth, a groove, and/or a lip, according to embodiments of the present invention. These retaining means may be formed by teeth or grooves arranged in an outer or inner face of the parts 21, or even by hooking means which are arranged in the vicinity of the distal ends 21A or proximal ends 21B of the parts 21, according to embodiments of the present invention.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A compression staple comprising:
a first arm element, the first arm element comprising:
a first anchor portion configured to be inserted into bone;
a first manipulation portion; and
a first breakaway feature between the first anchor portion and the first manipulation portion;
a second arm element, the second arm element comprising:
a second anchor portion configured to be inserted into bone;
a second manipulation portion; and
a second breakaway feature between the second anchor portion and the second manipulation portion; and
a bridge element spanning the first and second arm elements,
wherein moving the first manipulation portion away from the second manipulation portion moves the first anchor portion toward the second anchor portion.

2. The compression staple of claim 1, wherein a first longitudinal axis of the first arm element and a second longitudinal axis of the bridge element define a median plane, and wherein moving the first manipulation portion away from the second manipulation portion in a direction substantially parallel to the median plane moves the first anchor portion toward the second anchor portion.

3. The compression staple of claim 2, wherein applying a force to the first manipulation portion transverse to the median plane separates the first manipulation portion from the first anchor portion at the breakaway feature.

4. The compression staple of claim 1, wherein the first breakaway feature is a notch.

5. The compression staple of claim 1, wherein the first breakaway feature is a groove.

6. The compression staple of claim 1, wherein the first breakaway feature is a cutting guide.

7. The compression staple of claim 1, wherein a first thickness of the first arm at the first breakaway feature is less than a second thickness of the first manipulation portion near the first breakaway feature, and wherein the first thickness is also less than a third thickness of the first anchor portion near the first breakaway feature.

8. The compression staple of claim 1, wherein the first breakaway feature merges with second breakaway feature.

9. The compression staple of claim 1, wherein the bridge element includes an outward curvature toward the first and second anchor portions when the first and second arms are substantially parallel.

10. The compression staple of claim 9, wherein the outward curvature changes as the first anchor portion moves toward the second anchor portion.

11. The compression staple of claim 10, wherein the outward curvature lessens as the first anchor portion moves toward the second anchor portion.

12. The compression staple of claim 1, wherein the compression staple is integrally cut from a single metal plate.

13. A compression staple (1) of the type comprising two arms (2) connected by a transverse bridge (4), characterized in that each arm (2) comprises a first part (21) and a second part (23) arranged so as to extend from one another and connected by separable means (25), the first part (21) being used to anchor the arm (2) in a bone (9), whereas the second part (23) is used to guide the arm (2) in displacement relative to the bridge (4).

14. The compression staple of claim 13, characterized in that the second part (23) is used to guide the arm (2) in displacement relative to the bridge (4) and parallel to a median plane (π) defined by the longitudinal axis (X₂) of the arm (2) and the longitudinal axis (Y₄) of the bridge (4).

15. The compression staple of claim 13, characterized in that the bridge (4) is connected to each arm (2) at an end (21B) of the first part (21) adjacent to the second part (23).

16. The compression staple of claim 13, characterized in that the first part (21) and second part (23) of each arm (2) are connected by separable means (25) which can be broken off by exerting a force (F₄) transverse to the median plane (π) defined by the longitudinal axis (X₂) of the arm (2) and the longitudinal axis (Y₄) of the bridge (4).

17. The compression staple of claim 16, characterized in that the separable means of each arm (2) comprise a separable bridging connection (25) between the first part (21) and the second part (23) having a thickness (e₂₅), in a transverse direction (Z) relative to the median plane (π), which is smaller than the thickness (e₂₁, e₂₃) of the first and second parts in said direction (Z).

18. The compression staple of claim 17, characterized in that the separable bridging connection (25) of each arm (2) is defined by at least one notch (27) in the arm (2).

19. The compression staple of claim 13, characterized in that the first part (21) and second part (23) of each arm (2) are connected by separable means (25) which can be broken off by exerting a force (F₄) on either the first part or the second part of the arm, while the other part of the arm remains still.

20. The compression staple of claim 13, characterized in that the separable means (25) of each arm (2) define a cutting face (S₂₅) between the first part (21) and second part (23) of the arm.

21. The compression staple of claim 20, characterized in that the cutting face (S₂₅) of each arm (2) is arranged so the first part (21) of the arm (2) is inclined towards the first part (21) of the other arm (2) at the same height as at least a part of an edge (44) of the bridge (4) on the side of the second part (23) of each arm (2).

22. The compression staple of claim 13, characterized in that the end (21B) of the first part (21) of each arm (2) adjacent to the second part (23) of the arm has a cross-section (σ₁), perpendicular to the longitudinal axis (X₂) of the arm, which is greater than the cross-section (σ₂) of the rest of the first part (21) perpendicular to the longitudinal axis (X₂) of the arm.

23. The compression staple of claim 13, characterized in that before the compression staple (1) is implanted in a bone, the bridge (4) is curved outwardly on the side of the first parts (21) of the two arms (2).

24. The compression staple of claim 13, formed by a metal plate cut substantially into an H shape, each arm (2) of the H shape pivotable relative to the transverse bridge (4) of the H and parallel to the median plane (π) of the plate.

25. The compression staple of claim 13, characterized in that the second part (23) of each arm (2) comprises an aperture (24) for receiving an element for actuating displacement of the arm (2) relative to the bridge (4) and parallel to a median plane (π) defined by the longitudinal axis (X₂) of the arm (2) and the longitudinal axis (Y₄) of the bridge (4).

26. The compression staple of claim 13, characterized in that the first part (21) of each arm (2) comprises a means for holding the first part (21) in place when anchored in a bone (9).

27. A method for installing the compression staple of claim 1, comprising:
forming a first hole in a first bone part and a second hole in a second bone part;
inserting the first anchor portion into the first hole and the second anchor portion into the second hole until the bridge element contacts one or more of the first and second bone parts;
moving the first manipulation portion away from the second manipulation portion to compress the first and second bone parts between the first and second anchor portions; and
separating the first manipulation portion from the first anchor portion at the first breakaway feature and the second manipulation portion from the second anchor portion at the second breakaway feature.

28. The method of claim 27, further comprising:
moving the first manipulation portion toward the second manipulation portion to separate the first and second anchor portions to fit the first and second anchor portions into the first and second holes.
